(19) United States
(12) Patent Application Publication
(10) Pub. No.: US 2023/0414290 A1
(43) Pub. Date: Dec. 28, 2023

(54) METHODS FOR OPTIMIZATION OF PLACEMENT OF SPINAL CORD EPIDURAL STIMULATION UNIT

(71) Applicant: University of Louisville Research Foundation, Inc., Louisville, KY (US)

(72) Inventors: Susan J. Harkema, Louisville, KY (US); Samineh Mesbah, Louisville, KY (US); Maxwell Boakye, Louisville, KY (US); Claudia Angeli, Louisville, KY (US); Yangsheng Chen, Louisville, KY (US)

(73) Assignee: University of Louisville Research Foundation, Inc., Louisville, KY (US)

(21) Appl. No.: 18/253,780

(22) PCT Filed: Nov. 22, 2021
(86) PCT No.: PCT/US2021/060390

§ 371 Date: May 20, 2023

Related U.S. Application Data
(60) Provisional application No. 63/117,232, filed on Nov. 23, 2020.

(51) Int. Cl. A61B 34/10; G16H 30/40; G16H 20/30; G16H 50/50

(52) U.S. Cl. CPC A61B 34/10; G16H 30/40; G16H 20/30; A61N 1/0553; A61B 2034/105; A61B 2034/107; G16H 50/50

(57) ABSTRACT

Methods for optimization of surgical placement of an implantable electrode for spinal cord epidural stimulation of a subject include creating a computational model of the subject spinal cord based on medical imagery, determining the position of the lumbosacral enlargement, and determining an optimal placement to maximize volumetric coverage of the lumbosacral enlargement by the implantable electrode.

This application claims the benefit of U.S. provisional patent application Ser. No. 63/117,232 filed 23 Nov. 2020 for METHODS FOR OPTIMIZATION OF PLACEMENT OF SPINAL CORD EPIDURAL STIMULATION UNIT, incorporated herein by reference.

### TECHNICAL FIELD

Methods for optimization of surgical placement of an implantable electrode for spinal cord epidural stimulation of a subject include creating a computational model of the subject spinal cord based on medical imagery, determining the position of the lumbosacral enlargement, and determining an optimal placement to maximize volumetric coverage of the lumbosacral enlargement by the implantable electrode.

### BACKGROUND OF THE INVENTION

Spinal cord injury (SCI) is the second leading cause of paralysis in the United States with over 1.4 million individuals currently living with SCI related disabilities. Restoration of voluntary movement, bladder, bowel and sexual function are among the top priorities of the SCI population. To date there are no pharmacological treatments that have enabled voluntary movement in those completely paralyzed. In previous studies, some individuals that are diagnosed with clinically motor complete injuries have shown motor pool activation below the injury level during volitional attempts. However, the volitional descending inputs passing through the residual connections alone have not been shown to lead to eliciting movement. Moreover, studies have shown that engaging the sensory feedback via locomotor training interventions were also insufficient to promote sustained recovery of independent standing or stepping in the vast majority of clinically diagnosed motor complete cases.

Spinal cord epidural stimulation (scES) was shown to enable voluntary movement in completely motor paralyzed individuals following SCI. Subsequently, many reports confirmed scES ability to enable voluntary movements and standing and eventually stepping and walking over ground as well as improved cardiovascular function and bladder function in the chronic and diagnosed motor complete SCI population who did not have clinically detectable supraspinal influence.

The evidence of restoration of volitional motor function with scES has suggested that in the presence of scES with appropriate targeted electric fields, the spinal networks of the lumbosacral spinal cord would reach the excitability state needed to interpret the residual volitional descending inputs and sensory information to generate meaningful motor output. Spatially and temporally targeting the posterior roots of specific motor pools is another approach to facilitate motor recovery in incomplete SCI. However, there exists considerable variability regarding the extent of individuals' volitional recovery with epidural stimulation and many unidentified factors that influence an individual's response to epidural stimulation. There is considerable heterogeneity in the SCI population that can potentially affect volitional responses with scES including participants' age, duration of injury, type of injury, length of severe myelomalacia, amount of spinal cord atrophy after injury, size of the lumbosacral enlargement as well as final position of the electrodes in relation to the lumbosacral enlargement of the spinal cord.

The device implantation in individuals with chronic SCI currently involves placement of an electrode array, often a paddle electrode, at the lumbosacral segment of the spinal cord in a vertebral location typically between the T11 and L1 pedicles. However, the length of the spinal cord, the surrounding vertebral column, the location of the conus tip, and the relationship between the spinal cord levels and vertebral levels—particularly at the lumbosacral enlargement—is variable between individuals. Put another way, the spinal cord is hidden within the vertebral column, so a surgeon implanting a paddle electrode cannot readily confirm that aligning the paddle electrode with specific vertebral levels (which are visible during surgery) will result in the desired placement of the paddle electrode with respect to specific spinal cord levels (which are not visible during surgery). In current practice, a paddle electrode is initially placed in a subject per medical literature (i.e., the generally understood location of spinal cord levels with respect to vertebral levels), then the location of the paddle electrode is adjusted based on intra-operative fluoroscopy and electrophysiology mapping to identify segments of the spinal cord that respond best to electrical stimulation. Following the surgery, additional neurophysiological spatiotemporal mapping of various electrode configurations is used in combination with the motor task (e.g., voluntary leg movement, standing or stepping) to restore motor control in individuals with chronic motor complete injuries. However, this current practice does not account for spinal cord variability between individuals in the initial placement of the paddle electrode. Optimizing the initial placement of the implantable electrode would reduce, or in some cases eliminate, the need for interoperative adjustments to the location of the electrode, therefor reducing the duration of the surgery as well as potentially improving the subject's volitional recovery with scES.

### SUMMARY

The inventors reviewed magnetic resonance imaging (MRI), X-rays and clinical records of research participants that have undergone scES implantation to explore correlations between the amount of cervical cord atrophy above the injury, the length of the injury myelomalacia and the number of joints moved in the presence of scES. Correlations were also examined between number of joints moved and the amount of terminal lumbosacral enlargement coverage, and paddle position with respect to the tip of the conus and maximal cross-section area of the lumbosacral enlargement. The inventors found that individuals who had greater coverage of the lumbosacral enlargement by the paddle electrode exhibited greater number of joints moved. Accordingly, optimization of placement of the paddle electrode using MRI-driven measurements to maximize coverage of the lumbosacral enlargement by the paddle electrode is likely to improve the subject's volitional recovery with scES and guide optimal positioning of the paddle electrode for the best responses.

It will be appreciated that the various systems and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1 is a flowchart summarizing a method for optimizing electrode placement for spinal cord epidural stimulation.

FIG. 2 references integration of axial and sagittal MRIs with intra-operative fluoroscopy images to estimate the location of the scES paddle electrode on the spinal cord. Panel A displays exemplary MRI in sagittal view from Thoracic-Lumbar (T-L) level of the spine and corresponding axial MRI slices. Panel B depicts neurophysiological mapping of muscle responses to the stimulation at rostral and caudal parts of the array (black contact: cathode/−, grey contact: anode/+) and the corresponding fluoroscopy image indicating the location of the paddle electrode with respect to the bone structure, i.e. vertebral levels.

FIG. 3 references measurements of the injury site using MRI. Panel A displays an exemplary MRI image in sagittal view from cervical-thoracic (C-T) level of the spine and the corresponding axial MRI slices. Panel B displays a cross-sectional area of the spinal cord at the inferior endplate of C3 with segmented cord tissue in orange and spinal canal in red; the spinal canal including both the dura and CSF space within it, and the epidural fat. Panel C is an MRI image displaying the length of the injury calculated from the axial MRI slices with severe myelomalacia.

FIG. 4 references calculation of the cross-section areas and three-dimensional (3D) reconstruction of the spinal cord at lumbosacral enlargement from the axial MRI slices. Panel A depicts exemplary MRI slices in axial view with manual segmentation of the spinal cord tissue and the spinal canal highlighted in cyan and grey, respectively. Panel B is a chart depicting cross-section areas (CSA) of each segmented MRI slice for lumbosacral enlargement (LSE) tissue (lower line) and the spinal canal (upper line). The maximal CSA of lumbosacral enlargement, the tip of the conus, the distance (L) between the maximal CSA and the conus tip, and estimated beginning of the lumbosacral enlargement at distance 2L from the conus tip are each indicated, and the estimated total volume of the lumbosacral enlargement is shown by the shaded portion under the lower line. Panel C is a 3D reconstruction of the spinal cord at lumbosacral enlargement (shown in darker grey) from segmented MRI slices and the spinal canal represented in lighter grey.

FIG. 5 depicts, in panels A and B, for all participants, graphs of cross-section areas (CSA) of each segmented MRI slice for lumbosacral enlargement (LSE) tissue (lower line) and the spinal canal (upper line). The maximal CSA of lumbosacral enlargement, the tip of the conus, the distance (L) between the maximal CSA and the conus tip, and estimated beginning of the lumbosacral enlargement at distance 2L from the conus tip are indicated on each graph.

FIG. 6 depicts exemplary 3D reconstructed images of lumbosacral enlargement with paddle electrode placement and visualization of calculated measurements. Panel A is a 3D reconstructed lumbosacral enlargement for subject A60 with the distance L between the maximal enlargement slice (darker cyan) and conus tip and 2L distance from the conus tip to approximate beginning of the lumbosacral enlargement marked with dashed blue lines. Panel B is a visualization of the final placement of the paddle electrode on the lumbosacral enlargement for A60 with an estimated 79% of coverage. The top and end of the paddle electrode are marked with dashed black curves on the cord. Panel C displays the estimated total volume of the lumbosacral enlargement portion of the spinal cord in dark shading, with the subsection of the LSE volume covered by the electrode paddle indicated by wavy lines. The distances between the top of the paddle electrode and the maximal enlargement, and the caudal end of the paddle and the conus tip are indicated with dashed lines and blue arrows.

FIG. 7 is a series of charts depicting correlations between the radiographic measurements, i.e. the cord CSA at C3 above the injury, length of severe myelomalacia, estimated percentage of lumbosacral enlargement coverage, and the distances from paddle electrode to the maximal enlargement and conus tip, and the number of joints moved with scES. Panel A depicts the correlation between the cross-section area (CSA) of the spinal cord at the inferior endplate of C3 and number of joints moved post-operation for subjects with proper MRI imaging at this level (n=19). Panel B depicts the correlation between the length of the severe myelomalacia and number of joints moved post-operation for subjects with proper MRI imaging at this level (n=18). Panel C depicts the correlation between the percentage of the lumbosacral enlargement volume coverage by the scES paddle electrode and number of joints moved post-operation for all subjects (n=20). Panel D depicts the correlation between the sum of distances from the top of the scES paddle to the maximal enlargement the caudal end of the paddle to the conus tip, and number of joints moved post-operation for all subjects (n=20).

FIG. 8 is a graph depicting a correlation analysis for the subgroup of individuals with positive trend (N=16) between the number of joints moved volitionally and the percentage of lumbosacral enlargement coverage by scES.

FIG. 9 is a series of graphs depicting correlations between the number of joints moved voluntarily post-implant and demographic and neuroanatomical factors including (A) distance between the maximal cross-section area of the lumbosacral enlargement (LSE) and the conus tip, L; (B) maximal cross-section area of the spinal cord at LSE; (C) estimated total volume of the spinal cord at LSE; (D) participants' age at the time of implant; and (E) time since injury at the time of implant.

FIG. 10 depicts a 3D visualization of the spinal cord and relative paddle electrode placement on the lumbosacral enlargement. Panel A is a schematic representation of a Medtronic Specify® 5-6-5 lead paddle electrode as used for spinal cord epidural stimulation, reproduced from Medtronic Manual—permission granted from Medtronic Inc. Panel B is a visualization of reconstructed spinal cords of 20 research participants and the estimated paddle electrode placement with descending order of percentage of lumbosacral enlargement coverage from left to right and up to down.

FIG. 11 is a graph depicting a correlation between the percentage of lumbosacral enlargement coverage and sum of distances from the maximal enlargement and conus tip.

FIG. 12 is a schematic representation of neuroanatomical mapping of the spinal cord at the lumbosacral enlargement including (A) MRI sagittal view of the whole spine in an individual with cervical spinal cord injury; (B) axial images of lumbosacral region from high spatial resolution MRI recording with arrows pointing at dorsal nerve roots from the vertebral site of exit back to the location of entering the spinal cord to show how spinal cord levels are identified using nerve root tracing technique; (C) process of tracing and labeling spinal cord region, cerebrospinal canal and nerve roots in each axial image and using isosurface function to build the reconstructed model of lumbosacral spinal cord from segmented images; and (D) process of integrating intra-operative fluoroscopy images of the paddle placement with the pre-operative MRI recordings in order to estimate the location of the paddle array on the spinal cord model.

FIG. 13, in panels A and B, illustrates 3D computer models of lumbosacral spinal cord reconstruction, estimated electrode paddle placement, and location of vertebral bodies with respect to spinal cord levels for twelve participants.

FIG. 14 is a serial of graphs depicting neuroanatomical characteristics of the human spinal cord, including (A) a correlation plot between whole spine length and whole spinal cord length for 12 participants (n=12); (B) a correlation plot between whole spine length and lumbosacral enlargement and conus (LSE) length (n=12); (C) a correlation plot between whole spinal cord length and LSE (n=12); and (D) individual values and boxplots of average cross section areas of spinal cord at lumbosacral levels L1, L2, . . . L5 and sacral (S) region. Panels (A-C) include linear fitted lines and statistical values including Pearson's r, Adjusted R-square, Intercept, Slope and P-value.

### DETAILED DESCRIPTION OF THE PREFERRED EMBODIMENTS

For the purposes of promoting an understanding of the principles of the invention, reference will now be made to selected embodiments illustrated in the drawings and specific language will be used to describe the same. It will nevertheless be understood that no limitation of the scope of the invention is thereby intended; any alterations and further modifications of the described or illustrated embodiments, and any further applications of the principles of the invention as illustrated herein are contemplated as would normally occur to one skilled in the art to which the invention relates. At least one embodiment of the invention is shown in great detail, although it will be apparent to those skilled in the relevant art that some features or some combinations of features may not be shown for the sake of clarity.

Any reference to “invention” within this document is a reference to an embodiment of a family of inventions, with no single embodiment including features that are necessarily included in all embodiments, unless otherwise stated. Furthermore, although there may be references to “advantages” provided by some embodiments of the present invention, other embodiments may not include those same advantages, or may include different advantages. Any advantages described herein are not to be construed as limiting to any of the claims.

Specific quantities (spatial dimensions, dimensionless parameters, etc.) may be used explicitly or implicitly herein, such specific quantities are presented as examples only and are approximate values unless otherwise indicated. Any quantities referred to as “about” a given value are defined as being within 5% of the stated value unless otherwise specified (e.g., “about 1.0 mm” refers to the range of 0.95 mm to 1.05 mm, “between about 1.0 mm and 2.0 mm” refers to the range of 0.95 mm to 2.1 mm). Discussions pertaining to specific compositions of matter, if present, are presented as examples only and do not limit the applicability of other compositions of matter, especially other compositions of matter with similar properties, unless otherwise indicated. The terms top and bottom, upper and lower, left and right, and similar language used herein refer to the orientations as shown in the drawings.

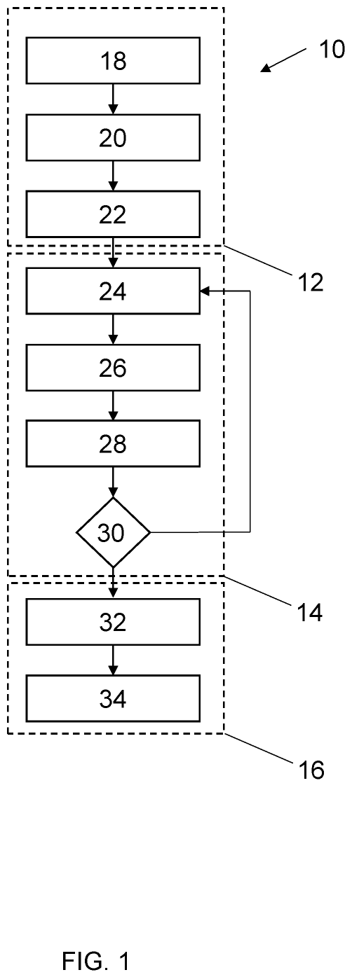

Referring now to FIG. 1, a method 10 for optimizing electrode placement for spinal cord epidural stimulation can be divided into pre-operative steps 12, intra-operative steps 14, and post-operative steps 16. Pre-operative steps 12 include a first step 18 of acquiring at least one medical image of the spinal cord and surrounding vertebral column, a second step 20 of anatomical mapping and construction of a computational 3D model of the spinal cord and surrounding vertebral column, and a third step 22 of predicting the optimal placement of the implantable electrode, such as a paddle electrode, based on the location of the electrode relative to the spinal cord levels and the corresponding coverage of the lumbosacral enlargement by the electrode. Intra-operative steps 14 include a fourth step 24 of initially placing the implantable electrode during implantation based at least in part on the prediction from step 22, a fifth step 26 of fluoroscopic imaging and neurophysiological mapping, and a sixth step 28 of acquiring at least one medical image of the spinal cord, surrounding vertebral column, and implantable electrode to determine the placement of the implantable electrode and percent coverage of the lumbosacral enlargement. The method then proceeds to decision step 30, in which, based at least in part on the mapping in step 26 and the determination in step 28, the practitioner decides whether to return to step 24 and adjust the positioning of the implantable electrode or proceed to step 32. Post-operative steps 16 include a seventh step 32 of post-implantation acquisition of at least one medical image of the final placement of the implantable electrode, and an eighth step 34 of integrating the 3D model and the at least one medical image acquired in the seventh step 32 to confirm the location of the implantable electrode with respect to spinal cord levels at the lumbosacral enlargement. Each of these steps are discussed in further detail below. In certain embodiments, the method may comprise only the pre-operative steps 12, namely, the first, second and third steps 18, 20, 22. In other embodiments, the method may comprise the pre-operative steps 12 and intra-operative steps 14. In further embodiments, the method may comprise the pre-operative steps 12, intra-operative steps 16, and post-operative steps 18.

scES has enabled volitional lower extremity movements in individuals with chronic and clinically motor complete SCI and no clinically detectable brain influence. The inventors found that the individuals' neuroanatomical characteristics or positioning of the scES electrode were important factors influencing the extent of initial recovery of lower limb voluntary movements in those with clinically motor complete paralysis. The inventors hypothesized that there would be significant correlations between the number of joints moved during attempts with scES prior to any training interventions and the amount of cervical cord atrophy above the injury, length of post-traumatic myelomalacia and the amount of volume coverage of lumbosacral enlargement by the stimulation electrode array. The clinical and imaging records of twenty individuals with chronic and clinically motor complete SCI who underwent scES implantation were reviewed and analyzed using MRI and X-ray integration, image segmentation and spinal cord volumetric reconstruction techniques. All individuals that participated in the scES study (N=20) achieved, to some extent, lower extremity voluntary movements post scES implant and prior to any locomotor, voluntary movement or cardiovascular training. The correlation results showed that neither the cross-section area of spinal cord at C3 (N=19, r=0.33, p=0.16) nor the length of severe myelomalacia (N=18, r=−0.02, p=0.93) correlated significantly with volitional lower limb movement ability. However, there was a significant, moderate correlation (N=20, r=0.58, p=0.007) between the estimated percentage of the lumbosacral enlargement coverage by the paddle electrode as well as the position of the paddle relative to the maximal lumbosacral enlargement and the conus tip (N=20, r=0.51, p=0.02) with the number of joints moved volitionally. These results suggest that greater coverage of the lumbosacral enlargement by scES may improve motor recovery prior to any training, possibly because of direct modulatory effects on the spinal networks that control lower extremity movements indicating the significant role of motor control at the level of the spinal cord.

Experimental design. Twenty participants were enrolled in research studies conducted at the University of Louisville investigating the effects of activity-based recovery training in combination with scES for lower limb motor function as well as studies investigating the use of scES for the restoration of cardiovascular regulation between 2014-2019. All research participants were over 21 years old at the time of implant with non-progressive SC above T10, AIS A or B, at least 2 years post injury, with functional segmental reflexes below the lesion and no clinically detectable brain influence on spinal reflexes. Moreover, none of the participants had any medical conditions unrelated to SCI during the scES implant and training. Table 1 shows demographic and clinical characteristics of the research participants.

TABLE 1
Measurements of the cord atrophy above the injury site (CSA at the inferior
endplate of C3) and the length of severe myelomalasia on the injury site:

| | Cross- | Cross- | | | MRI Slice |
|---|---|---|---|---|---|
| | section area | section area | Beginning | | thickness at |
| | at the inferior | at the inferior | and ending | length of | the Cervical- |
| | endplate | endplate | axial slices | severe | Thoracic |
| Publication | of C3 | of C3 | with severe | myelomalasia | level |
| ID | Rater #1 | Rater #2 | myelomalasia | (mm) | (mm) |
| A59 | 56 | 65 | N/A | N/A | 2.3 |
| B23 | 54 | 66 | 11/12-15 | 12.08 | 3.45 |
| A41 | 50 | 54 | 10-22 | 41.40 | 3.45 |
| A60 | 61 | 56 | N/A | N/A | 3.45 |
| A68 | 41 | 39 | 10-20 | 34.50 | 3.45 |
| B21 | 43 | 51 | 10-22 | 41.40 | 3.45 |
| A77 | 48 | 60 | 26-37 | 33.00 | 3 |
| A80 | 27 | 43 | 20-32 | 36.00 | 3 |
| B30 | 57 | 63 | 39-42 | 9.00 | 3 |
| A96 | 36 | 48 | 22-36 | 42.00 | 3 |
| A99 | N/A | N/A | 24-43 | 57.00 | 3 |
| A101 | 51 | 58 | 23-26 | 9.00 | 3 |
| B32 | 35 | 44 | 23-31 | 27.60 | 3.45 |
| A105 | 46 | 49 | 26-39 | 39.00 | 3 |
| B24 | 55 | 68 | 36-50 | 42.00 | 3 |
| B41 | 63 | 68 | 43-52 | 27.00 | 3 |
| A110 | 51 | 63 | 37-46 | 27.00 | 3 |
| A82 | 38 | 55 | 30-41 | 33.00 | 3 |
| A100 | 47 | 50 | 27-46 | 57.00 | 3 |
| A123 | 37 | 36 | 29-38 | 27.00 | 3 |

| Inter-rater reliability, ICC and 95% CI | 0.77 [0.66, 0.85] Good |
|---|---|
| Difference in | R_{rater#1} = 0.33 |
| Correlations | R_{rater#2} = 0.25 |
| with # of | R_{diff} = 0.07 |
| joints moved | [−0.22, 0.31] |
| and 95% CI | Difference is Not significant |

N/A: Could not be measured from the imaging records.

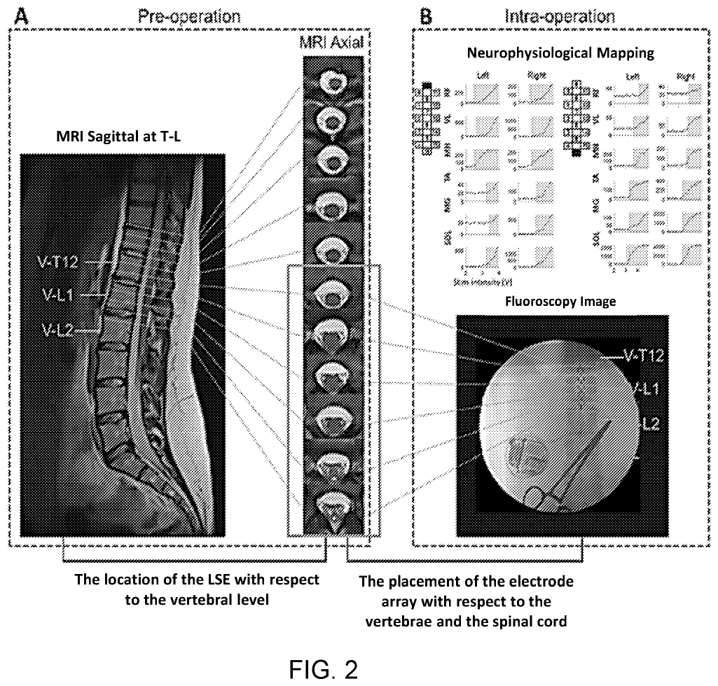

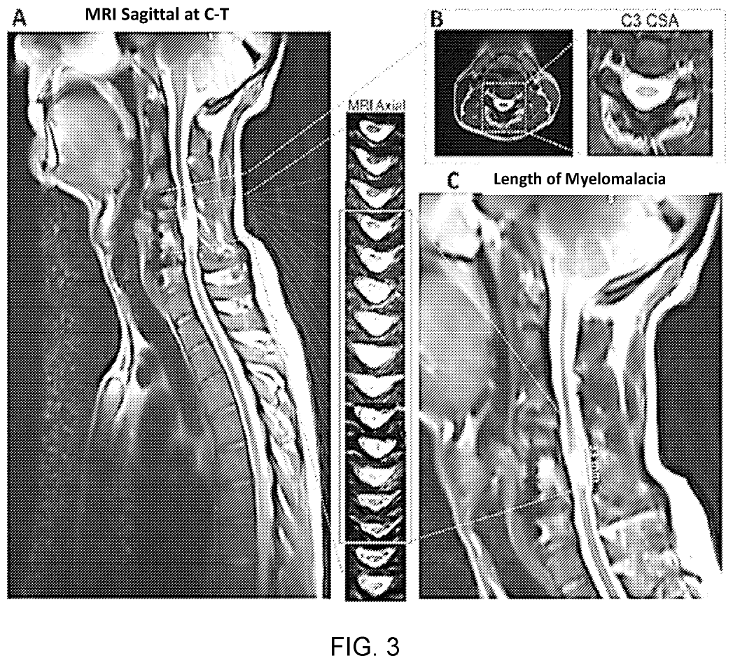

Pre-operative MRI recordings (first step 18). Prior to the implantation surgery, 3D magnetic resonance (MR) images from cervical-thoracic (C-T) and thoracic-lumbar (T-L) levels of the spinal cord in axial and sagittal views were recorded (FIG. 2, Panel A and FIG. 3). The images were obtained using a Siemens 3 Tesla system (Siemens Magnetom Skyra, Siemens Medical Solutions, Malvern, PA USA) with Turbo Spin Echo and T2-weighted pulse sequences.

With respect to the details of the MRI acquisition protocol, sagittal images were first obtained in two or three separate sequences to cover the spine from at least the foramen magnum to the mid lumbar or sacral regions with large field of view (FOV) images to screen patients for syrinxes, significant stenoses, scoliosis, levels of injury and stabilizing treatment-related surgical changes. Usually this was performed with two sequences, but taller subjects required three separate sequences because of field of view limitations. Typical parameters were:

TR/TE/FA/Thick/ETL/Re_Matrix/PFOV/NSA/BW/Pixal/AQ_matrix/% samp/PE=

Upper sagittal: 3000/74/160/3×3.45/17/320×320/100%/2/600/1.125×1.125/320×240/75/442

Lower sagittal: 3000/74/˜130/3×3.45/17/320×320/100/2/600/1.25×1.25/324×240/75/442

Where TR=repetition time, TE=echo time, Thick=slice thickness, ETL=echo train length, Re_Matrix=reconstruction matrix, PFOV=% phase field of view, NSA=number of signal averages, BW=bandwidth, Pixal=pixel dimensions, AQ_matrix=acquisition matrix, % samp=% sampling (or partial Fourier) and PE=number of phase 3 encodes. In a few cases, minor adjustments were made to the parameters for specific absorption rate (SAR) limitations, patient size, or clinical factors. Additional sequential axial T2 Turbo spin echo images were obtained from the foramen magnum to the T3-4 level. In most cases, these images were obtained either with a 10% gap (standard) or no gap. Axial images were obtained through thoracic and lumbar regions usually at 5 mm thickness with a 5 mm gap (or 3.45 mm thickness with a 0 mm gap) between images in order to reach sustainable imaging times that could be tolerated by the research participants. Example parameters are:

Cervical spine: 5190/74/160/3×3/26/512×512/100/2/610/0.35×0.35/256×179/70/260

Thoracic spine: 3690/82/121/5×10/28/512×512/100/2/610/0.35×0.35/256×179/70/252

Lumbar spine: 3280/82/121/5×10/28/512×512/100/2/610/0.35×0.35/256×179/70/252

Intra-operative procedure for scES paddle placement (fourth step 24, fifth step 26, sixth step 28, decision step 30, and seventh step 32). During the implantation procedure, anterior-posterior (AP) and lateral fluoroscopy imaging was used to mark the desired vertebral level with the participant in a prone position. A midline bilateral laminotomy was performed, typically at the L1-L2 disc space. The electrode array with 16 contacts (Medtronic Specify® 5-6-5 lead) was advanced into the epidural space via the laminotomy. AP fluoroscopy was used to confirm that the array was as symmetric as possible with respect to the midline. Electrophysiological mapping was performed after initial placement to optimize the positioning of the paddle electrode based on the evoked responses recorded by surface electrodes from soleus (SOL), medial gastrocnemius (MG), tibialis anterior (TA), medial hamstrings (MH), vastus lateralis (VL) and rectus femoris (RF) (FIG. 2, Panel B). Midline local rostral and caudal electrode configurations at 2 Hz, 450 μsec were tested to examine the sequence of activation of lower extremity muscles as the stimulation intensity was increased from 0.1 V or 0.5 V with 0.1 V until all muscles reached motor thresholds. The symmetry between left and right sides was evaluated by testing unilateral electrode configurations. If the sequence of muscle activations or the left and right symmetry was not satisfactory, the placement of the electrode array was adjusted, and the mapping process repeated until proper positioning was reached. Following each adjustment of the electrode array, lateral and/or AP fluoroscopy images were taken (FIG. 2, Panel B). After final placement was confirmed, the electrode lead was tunneled subcutaneously and connected to a neurostimulator. Lateral and AP X-ray images were obtained post-operation to record the final placement of the paddle electrode.

Evaluation of number of joints moved post implant. Participants began the experimental sessions in the laboratory after approximately 2-3 weeks from the surgical implantation of the spinal cord epidural stimulation unit, depending on the time course of wound healing and after clinical clearance was provided by the study neurosurgeon. A series of spatiotemporal mapping experiments in supine position were initially performed. Initial mapping was followed by the selection of scES parameters to facilitate volitional lower limb movements and evaluated by the number of joints moved. All these experimental sessions were carried out prior to any training.

Spatiotemporal mapping. The first step post-operation was to perform the spatiotemporal mapping experiments in supine position. During the mapping session, bipolar electrode stimulation using a single adjacent anode and cathode as well as wide field configurations (non-adjacent and multiple anode and cathode combinations) were selected with the pulse width of 450 or 1000 μs. The intensity of the stimulation ramped up stepwise from low to high at low frequency (2 Hz) or high frequency (30 Hz) as well as the frequency ramp up with the intensity fixed at supra threshold. The electromyogram (EMG) signals were recorded from 16 leg muscles including right (R) and left (L) gluteus maximus (GL), medial hamstring (MH), rectus femoris (RF), vastus lateralis (VL), tibialis anterior (TA), medial gastrocnemius (MG), soleus (SOL) and iliopsoas (IL). The participants did not perform any voluntary movement during the mapping sessions. The outputs of these neurophysiological spatiotemporal mappings would allow identification of the response thresholds and regional responses of each muscle to the stimulation and the segmental location of extensor and flexor muscle groups on the electrode array. These mappings also allowed identification of the fields that generated rhythmic locomotor activity.

Selection of scES parameters and assessment of volitional movement ability. Lower extremity voluntary movement configurations were then initially estimated using the data obtained from the spatiotemporal mapping sessions. Subsequent assessments in a supine position were performed using the estimated initial configurations while participants attempted voluntary movements. Configuration parameters were then optimized during voluntary movements with the goal of achieving the best movement pattern that promoted greatest range of motion. Three different antigravity joint movements were attempted bilaterally (total of six joints). Movements included hallux extension, ankle dorsiflexion and knee to chest flexion. The optimal configuration resulted in stimulation amplitudes that remained sub-motor threshold when not attempting to move and would promote the best motor output of agonist muscles during attempts. The search of configuration parameters could take 1-5 days and it was performed with and without EMG recording. The number of joints moved (0-6) was determined by the ability to successfully find scES configurations that allowed a minimum of 3 repetitions of the desired movement under command. This ability was demonstrated prior to any scES training. The performance of voluntary movement experiments was done blinded from the findings of this study. Moreover, the radiographic analysis performed in this study was blinded from the volitional motor outcomes of the research participants.

Spinal cord atrophy and lesion size. Two measurements at the injury site were considered for correlation analysis: The first measurement was the cross-section area (CSA) of the spinal cord at the inferior endplate of C3 vertebra that is above the injury site for most of our participants (N=19). Amount of area reduction at this level is an indication of the amount of spinal cord atrophy after SCI. To calculate this measurement, the area of the spinal cord at this level was manually segmented from the corresponding C-L axial MRI slice (FIG. 3, Panels A and B). The segmentation task was performed by two independent trained raters in order to measure the inter-rater variability and ensure that the amount of inter-rater variability does not interfere with the findings.

The second measurement is the length of severe myelomalacia of the cord at the site of injury that was calculated from the C-L axial and sagittal MRI slices for all subjects with proper imaging at the site of injury (N=18) (FIG. 3, Panels A and C). This measurement was performed by one trained rater based on counting the number of consecutive axial slices that show severe myelomalacia at the site of injury and the length of severe myelomalacia was calculated by multiplying the number of selected slices by the slice thickness.

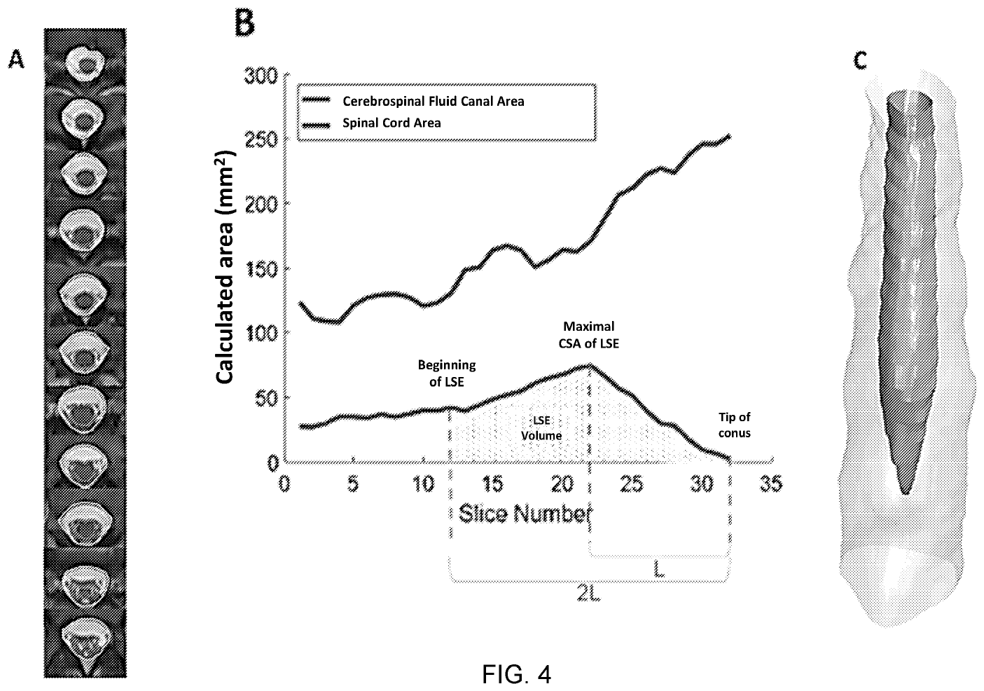

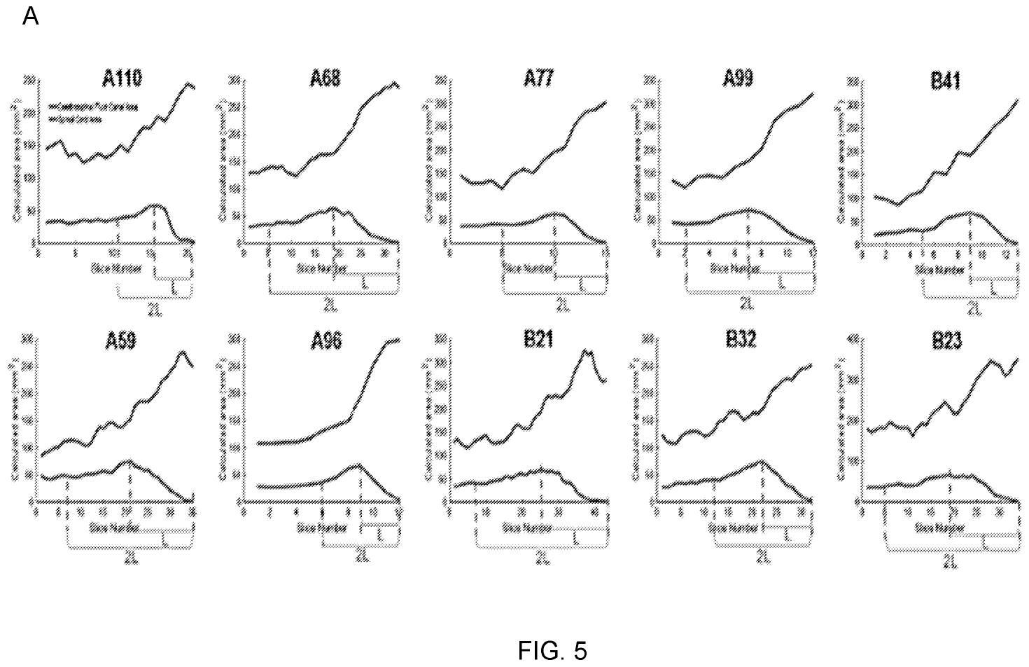

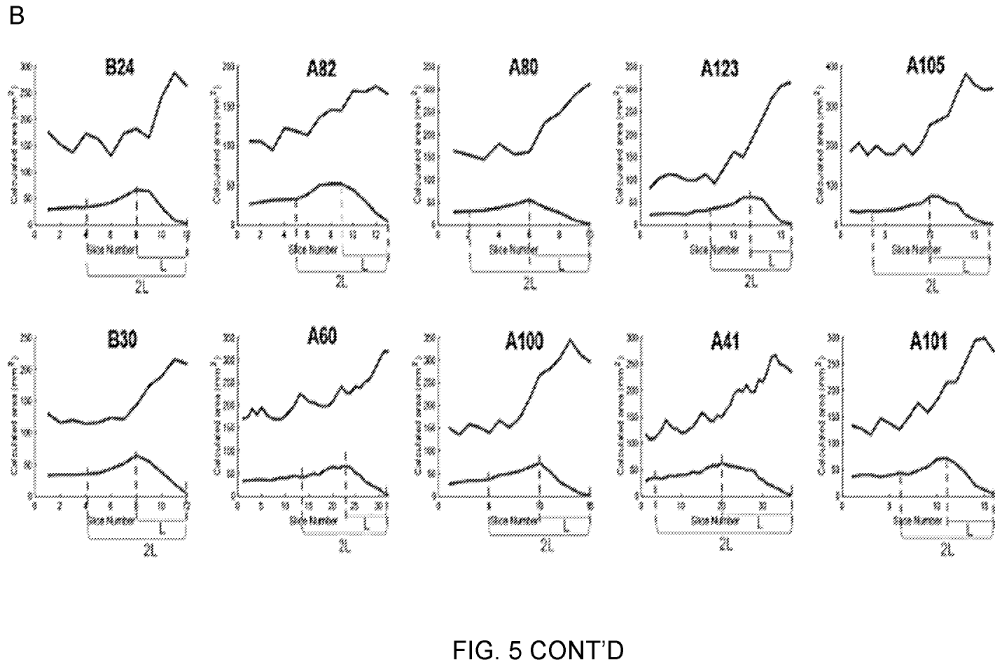

Size of the lumbosacral enlargement. To estimate the volume of lumbosacral enlargement above the conus tip, first, the cord (and the spinal canal) area from the axial MRI slices at T10-L1 level were segmented (FIG. 4, Panel A). The slices with the maximal CSA at the lumbosacral enlargement and the location of the tip of the conus were then identified and these two measurements used as reference points. The distance (L) between these two reference points was calculated, and an estimation of the beginning of the enlargement was marked as 2L distance above the conus tip (FIG. 4, Panel B). The volume of the lumbosacral enlargement was then calculated as the volume of the spinal cord from the 2L position to the conus tip in all subjects (FIG. 5). Finding the precise beginning of the lumbosacral enlargement is difficult and relatively subjective. The 2L estimation ensures a more consistent and objective measurement across subjects.

Once the estimated beginning of the lumbosacral enlargement is marked, the total volume of the lumbosacral enlargement and conus from the segmented cord CSAs, i.e. the area under the curve in FIG. 4, Panel B, was calculated. Finally, a 3D visualization of the lumbosacral enlargement from the segmented MRI slices in coronal view, shown in FIG. 4, Panel C, was reconstructed. Since the ‘true’ maximal CSA at enlargement might be between two consecutive MRI slices, the maximum error range for calculating lumbosacral enlargement volume was measured by selecting one slice above and one slice below the estimated beginning point of the lumbosacral enlargement and reporting the average values and standard deviations. In the 3D visualization, the major diameter of the maximal enlargement was calculated and used as reference for more accurate positioning of the scES paddle electrode on the lumbosacral enlargement.

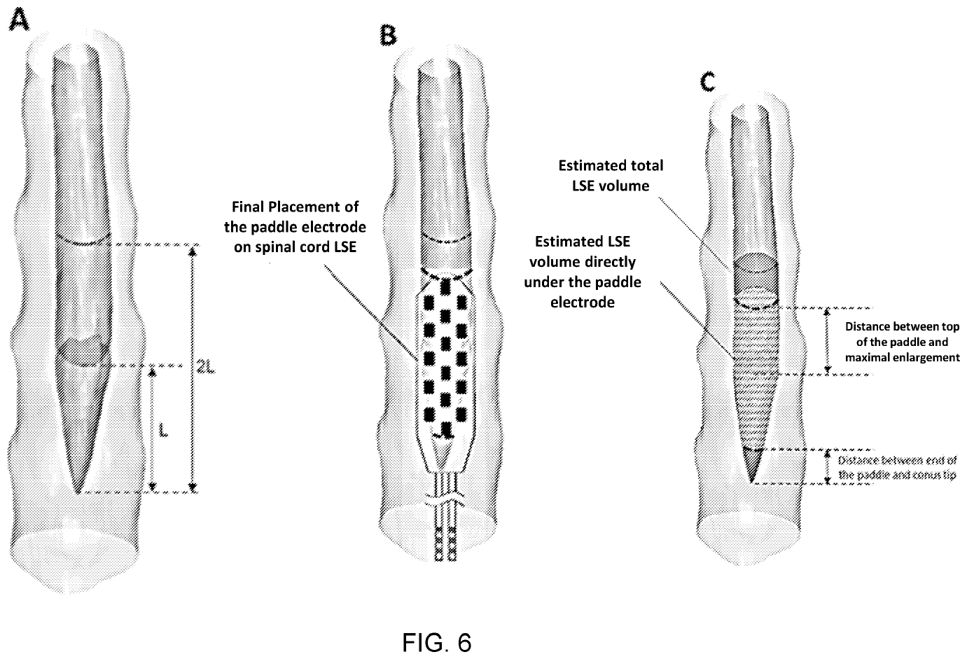

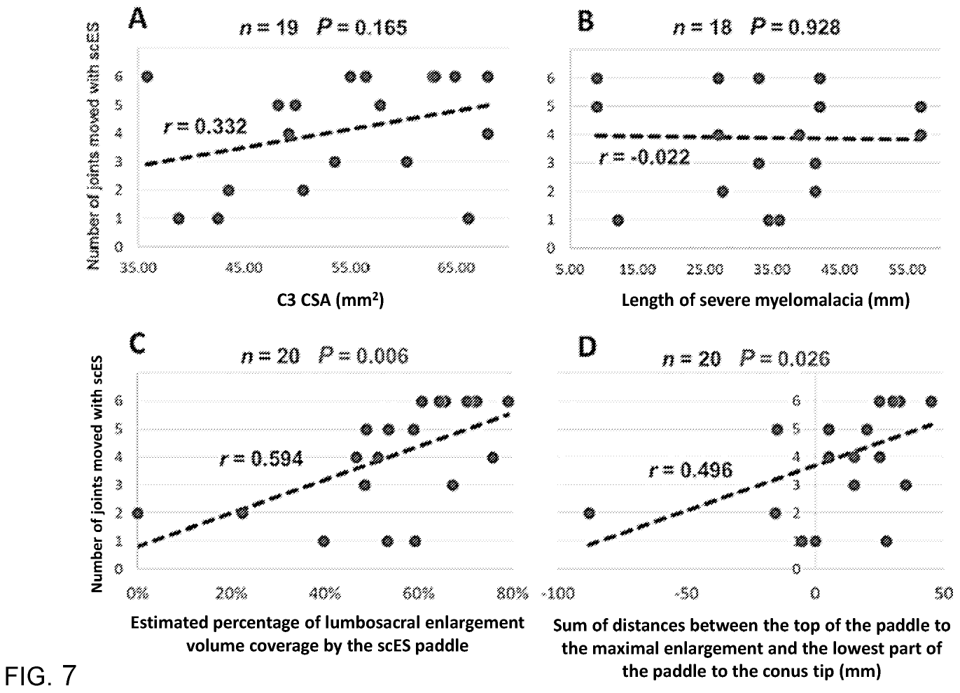

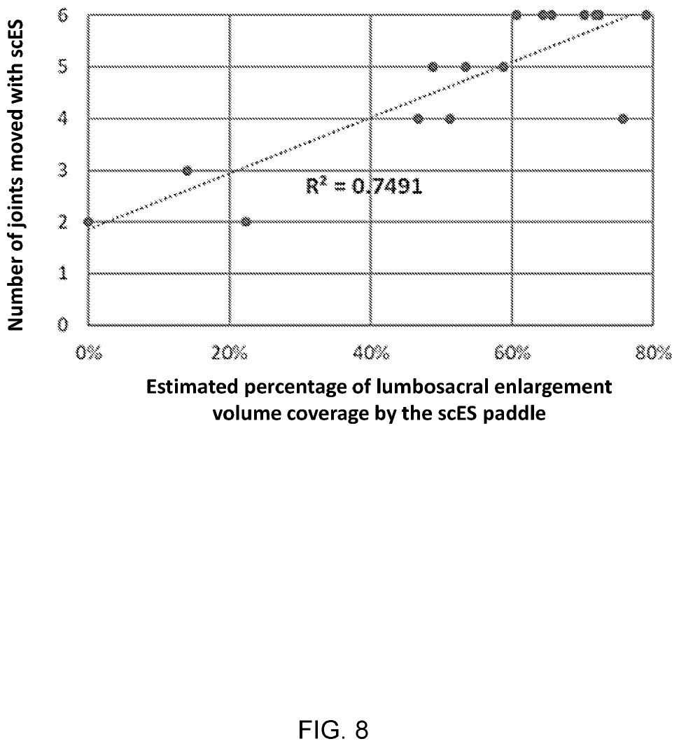

Spinal cord lumbosacral volume coverage by the electrode array. In order to estimate the volume of lumbosacral enlargement located under the implanted scES paddle electrode, the axial and sagittal MRIs were integrated with the X-ray images of the final placement of the electrode array. The location of the electrode contacts with respect to the vertebral level was determined using the X-ray, and the amount of cord tissue that is directly covered by the paddle electrode was estimated using axial MRI slices (FIG. 8). To minimize the interference of the angle of the fluoroscopy images in AP view with the accurate assessment of the lumbosacral enlargement coverage, the post-surgery X-ray images in lateral view were mostly used for this measurement. Once the right set of axial slices are selected, the amount of covered cord volume was estimated and the ratio of the lumbosacral enlargement under the paddle to the estimated total volume of the lumbosacral enlargement was reported as a percentage, i.e. estimated percentage of lumbosacral coverage by the scES paddle (FIG. 6, Panel C; FIG. 7, Panel C).

The estimated percentage of lumbosacral enlargement volume coverage is consequently related to the positioning of the electrode array with respect to the slices with maximal enlargement and the tip of the conus. Therefore, we also estimated the distance (mm) from the top of the electrode contact at the top of the paddle to the maximal enlargement slice and the distance from the caudal end of the electrode contact at the bottom of the paddle to the tip of the conus. If the paddle is above these two points of reference, the distances were reported as positive values and if below the two reference points, the distances were reported as negative values (FIG. 7, Panel D).

Statistical analyses. The statistical significance of correlation values was tested using Pearson's correlation test and p values less than 0.05 considered as significant. Inter-rater reliability was measured using the intra-class correlation coefficient (ICC). To test its significance, 1000 bootstrap samples were constructed to obtain the non-parametric 95% confidence interval built with the 2.5th percentile and 97.5th percentile of the resulting distribution. The ICC values were classified as either poor (<0.5), moderate (0.5-0.75), good (0.75-0.9).

All MRI segmentations were done manually using MANGO image processing software (ric.uthscsa.edu/mango). Two blinded independent raters performed all the segmentations. The measurements of CSA and volume of the cord and 3D reconstruction were performed using custom-written codes in MATLAB 2017b. The correlation graphs, linear trend lines, ICC and correlation coefficient values were generated in SAS (SAS Institute Inc.) and Microsoft Excel 2016.

The imaging and clinical records of 20 research participants implanted with scES were reviewed. There were 15 males and 5 females, 14 with American Spinal Injury Association Impairment Scale (AIS) A and 6 with AIS B. Neurological level of injury ranged from C3 to T4. All individuals were diagnosed as clinically motor complete and the mean duration of injury was 6.4 years ranging from 2.4 to 16.6 years (Table 1).

All individuals achieved voluntary movements after epidural stimulation implantation and prior to any training intervention in the presence of scES. The stimulation parameters, i.e. paddle electrode contacts configuration, intensity, frequency and pulse width, were optimized during voluntary movements for each individual with the goal of achieving the best possible movement pattern and maximum number of joints moved. The number of joints (left and right toe, ankle and hip joints, maximum of 6) moved for each participant are displayed in Table 1.

FIG. 7 includes four graphs illustrating the correlations between calculated radiographic measurements and the lower extremity motor outcomes in the presence of scES. The two measurements of the cord CSA at C3, i.e. above the lesion, (N=19, r=0.33, p=0.165) and the length of severe myelomalacia (N=18, r=−0.02, p=0.928) did not significantly correlate with the number of joints moved with scES (FIG. 7, Panels A and B). However, the percentage of the cord at lumbosacral enlargement covered by the paddle electrode (N=20, r=0.58, p=0.007) showed a moderate significant correlation with the number of joints moved with scES (FIG. 7, Panel C). Moreover, since the percentage of the lumbosacral enlargement coverage is linked to the distance between the paddle electrode and the two points of reference, i.e. maximal lumbosacral enlargement area and tip of the conus, we measured the amount of correlations between the sum of these two distances and the volitional responses. As shown in FIG. 7, Panel D, the correlation between the sums of distances from the paddle to the maximal enlargement and conus tip (N=20, r=0.513, p=0.020) are also significantly correlated with the number of joints moved. Radiographic measurements for all individuals are reported in Tables 1-3.

TABLE 2
Measurements of size of the lumbosacral enlargement (LSE) including the distance
between the maximal cross-section area of the spinal cord at lumbosacral enlargement
and the conus tip, and estimated total volume of the lumbosacral enlargement:

| | | | Distance | Estimated | |
|---|---|---|---|---|---|
| | Slice | | between the | volume of the | MRI axial |
| | number | Axial slice | maximal | spinal cord at | slice |
| | with | number for | cross-section | LSE - Average ± | thickness at |
| | maximal | the | area of LSE and | standard | Thoracic- |
| Publication | cross-section | estimated | the conus tip, L | deviation | Lumbar level |
| ID | area at LSE | conus tip | (mm) | (mm³) | (mm) |
| A59 | 21 | 34/35 | 47 | 4680 ± 166 | 3.45 |
| B23 | 19 | 33/34 | 50 | 4695 ± 126 | 3.45 |
| A41 | 20 | 38 | 62 | 4811 ± 103 | 3.45 |
| A60 | 23 | 34/35 | 40 | 2971 ± 148 | 3.45 |
| A68 | 19 | 33 | 48 | 3742 ± 114 | 3.45 |
| B21 | 25 | 42/43 | 60 | 5500 ± 143 | 3.45 |
| A77 | 10 | 14 | 40 | 4510 ± 389 | 10 |
| A80 | 6 | 9/10 | 35 | 2973 ± 166 | 10 |
| B30 | 8 | 12 | 40 | 3759 ± 335 | 10 |
| A96 | 9 | 11/12 | 25 | 3041 ± 309 | 10 |
| A99 | 7 | 11/12 | 45 | 5034 ± 253 | 10 |
| A101 | 11 | 15/16 | 45 | 5065 ± 375 | 10 |
| B32 | 22 | 31/32 | 33 | 3348 ± 138 | 3.45 |
| A105 | 10 | 16/17 | 65 | 6243 ± 346 | 10 |
| B24 | 8 | 11/12 | 35 | 3726 ± 322 | 10 |
| B41 | 9 | 12/13 | 35 | 3886 ± 289 | 10 |
| A110 | 16 | 19/20 | 35 | 4017 ± 346 | 10 |
| A82 | 8 | 13 | 50 | 4094 ± 273 | 10 |
| A100 | 10 | 14/15 | 45 | 4660 ± 333 | 10 |
| A123 | 12 | 16 | 40 | 3734 ± 315 | 10 |

TABLE 3
Measurements of the coverage of the lumbosacral enlargement
with epidural stimulation paddle electrode:

| | | | | Percentage | Percentage |
|---|---|---|---|---|---|
| | Beginning and | Distance | Distance | coverage of LSE | coverage of LSE |
| | ending slice | from the top | from end of | by paddle | by paddle |
| | numbers for | of the array | the paddle | electrode - | electrode - |
| | paddle | to the | contacts to | average ± | average ± |
| | electrode | maximal CSA | the conus | standard | standard |
| Publication | contacts | at LSE | tip | deviation | deviation |
| ID | placement | (mm) | (mm) | Rater #1 | Rater #2 |
| A59 | 16-30 | 17 | 15 | 61 ± 2% | 69 ± 2% |
| B23 | 20-34 | −3 | −2 | 40 ± 1% | 38 ± 1% |
| A41 | 28-42 | −28 | −14 | 14 ± 0% | 16 ± 0% |
| A60 | 17-31 | 21 | 12 | 79 ± 4% | 72 ± 3% |
| A68 | 15-29 | 14 | 14 | 59 ± 2% | 61 ± 2% |
| B21 | 29-43 | −14 | −2 | 22 ± 1% | 23 ± 1% |
| A77 | 8-12/13 | 20 | 15 | 67 ± 6% | 71 ± 6% |
| A80 | 5/6-10 | 5 | −5 | 53 ± 3% | 51 ± 4% |
| B30 | 6-11 | 20 | 10 | 72 ± 6% | 78 ± 7% |
| A96 | 8/9-13/14 | 5 | −20 | 53 ± 5% | 43 ± 3% |
| A99 | 6/7-11/12 | 5 | 0 | 51 ± 3% | 53 ± 3% |
| A101 | 10/11-15/16 | 5 | 0 | 49 ± 4% | 41 ± 3% |
| B32 | Off the cord | −38 | −50 | 0% | 0% |
| A105 | 10-15 | 0 | 15 | 47 ± 3% | 54 ± 4% |
| B24 | 6-11 | 20 | 5 | 72 ± 6% | 70 ± 7% |
| B41 | 7-12 | 20 | 5 | 76 ± 6% | 76 ± 5% |
| A110 | 13/14-18/19 | 25 | 20 | 66 ± 6% | 65 ± 5% |
| A82 | 6/7-11/12 | 15 | 10 | 64 ± 4% | 73 ± 6% |
| A100 | 9-13/14 | 10 | 10 | 59 ± 4% | 47 ± 3% |
| A123 | 10-15 | 20 | 10 | 70 ± 6% | 62 ± 4% |

| Inter-rater reliability, ICC and 95% CI | 0.98 [0.94, 0.99] Excellent |
|---|---|
| Difference in | R_{rater#1} = 0.59 |
| Correlations | R_{rater#2} = 0.55 |
| with # of | R_{diff} = 0.04 |
| joints moved | [−0.03, 0.13] |
| and 95% CI | Difference is Not significant |

Due to the uneven distribution of the data around the linear correlation line in FIG. 7, Panels C and D, this correlation was retested in the subgroup of research participants that follow the positive linear trend (N=16) which showed that similar linear correlation still holds but with much higher correlation coefficient of r=0.86 and p<0.0001 (FIG. 8). For the remaining 4 individuals, the negative trend means that despite having relatively fair coverage they did not move the expected number of joints based on the trend observed in the subgroup: subject B23 had 40% coverage, which is more than half of the highest percent coverage reported (79%), but only moved one joint, same for A80 and A68 with respectively 53% and 59% coverages that only moved one joint and A77 with 67% coverage that did not move more than half of their joints.

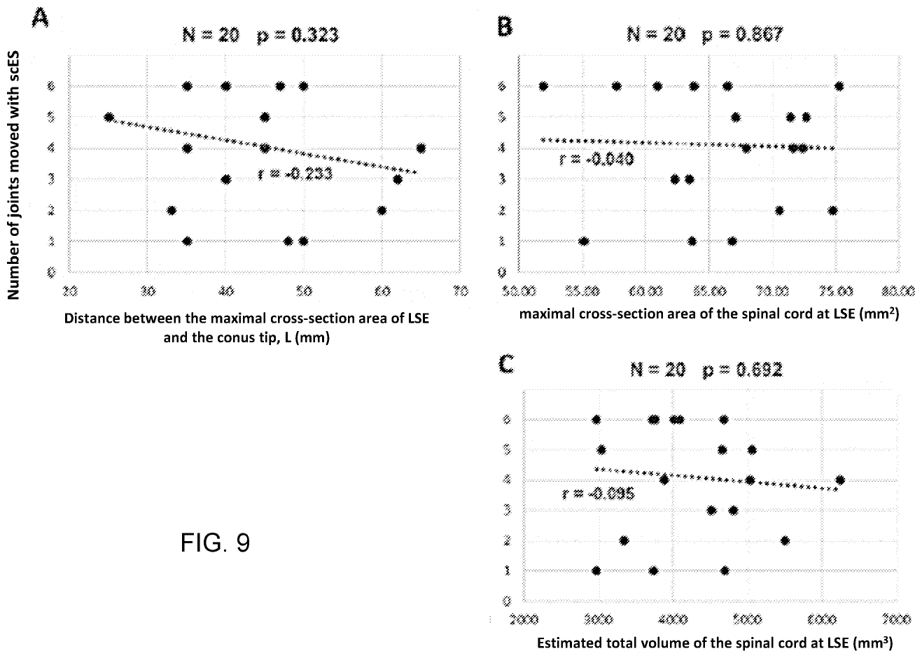

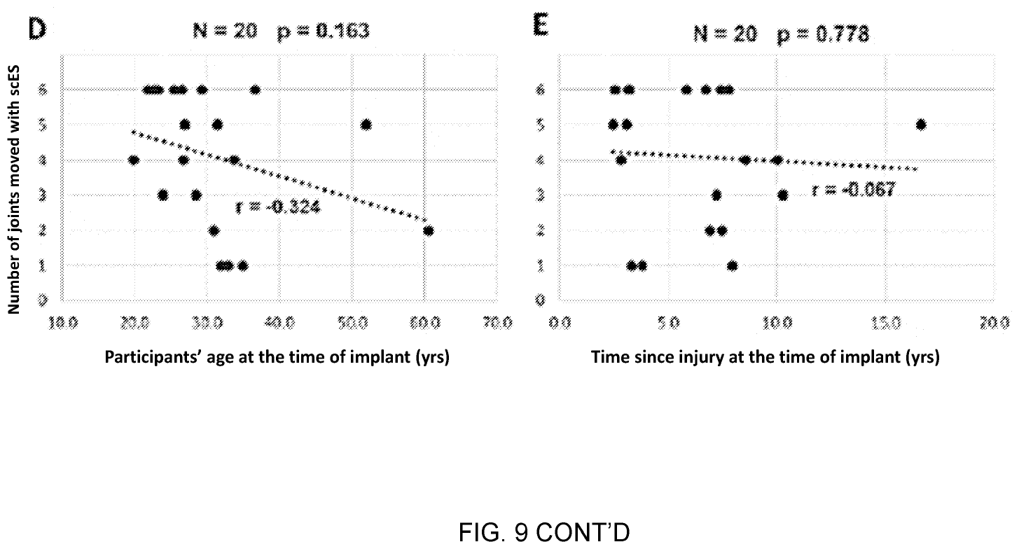

Correlations between other demographic and clinical factors such as age, time since injury, maximal enlargement CSA, the estimated total volume of lumbosacral enlargement, and the distance between the maximal enlargement and conus tip, with the number of joints moved voluntarily post-operation were also examined and none of these factors showed significant correlations (FIG. 9, Panels A-E).

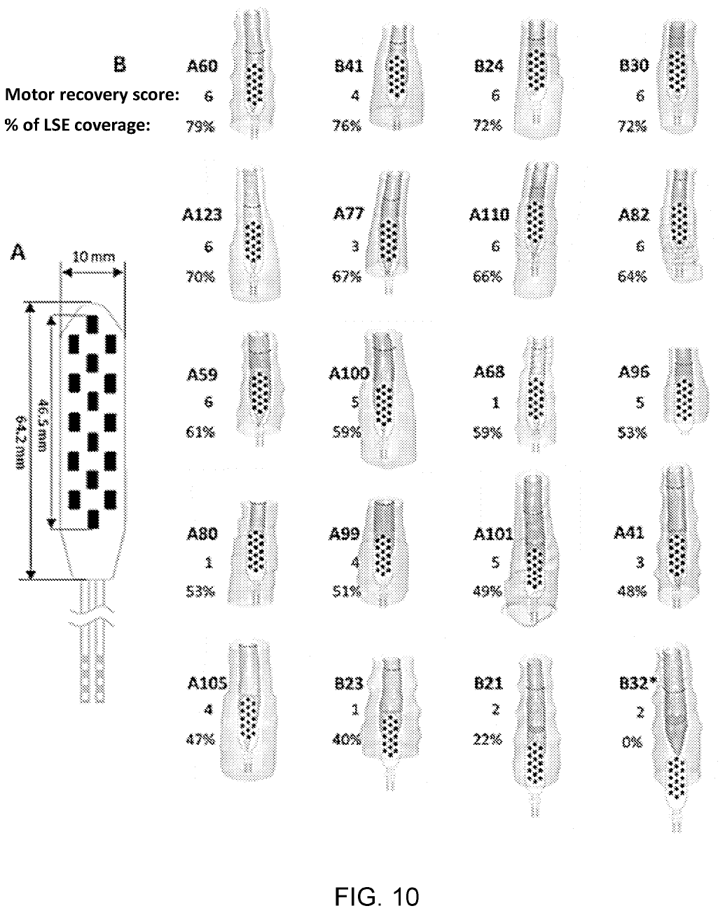

In order to visualize the amount of variability of the electrode array placement among the 20 participants, the results of the volumetric reconstruction of each participant's spinal cord at lumbosacral enlargement with the graphic of the paddle electrode placement over the dura and the acute motor function recovery scores are illustrated in FIG. 10. In these graphs, the width of the paddle electrode and the width of the maximal lumbosacral enlargement slice were considered to better represent the electrode positioning.

The inter-rater reliability was tested for spinal cord CSA above injury and lumbosacral enlargement percentage coverage values and the results showed that the ICC values were “good” and “excellent” for these measurements, respectively. Moreover, for both measurements, the difference between the Pearson's correlation coefficients of the two raters were not statistically significant (Tables 1 and 3).

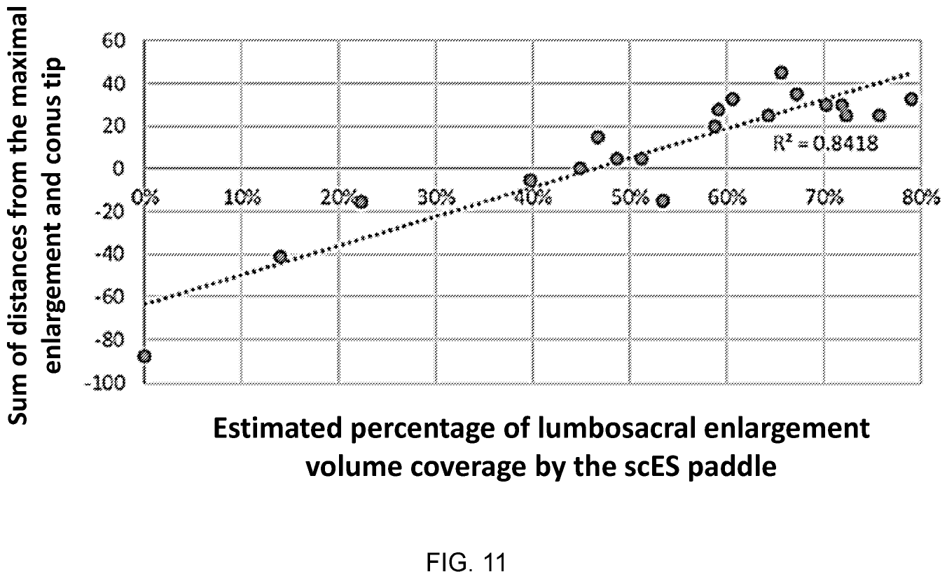

Finally, we tested the correlation between the two measurements for paddle placement on lumbosacral enlargement: the percentage of lumbosacral coverage and the positioning of the paddle electrode based on the distances between top of the array and maximal enlargement and bottom of the array and end of conus and showed that these two measures are “very highly” correlated (r=0.92, p<0.0001, N=20, FIG. 11). We also performed sensitivity analysis to compare the Pearson's correlation values of these two paddle placement measurements with the volitional outcomes and found that with p≤0.0001 these two measurements are statistically comparable. This analysis shows that the correlation results found in this study (FIG. 7, Panels C and D) are not sensitive to the choice of the measure used. This also assures that the selection of 2L distance as an estimation of the beginning of lumbosacral enlargement adds negligible error to the correlation analysis outcomes.

All individuals with chronic and clinically motor complete SCI that participated in the scES experiment (N=20) achieved, to some extent, lower extremity voluntary movements post scES implant and prior to any locomotor, voluntary movement or cardiovascular training. The accompanying figures show that number of joints moved voluntarily post-implant was significantly correlated with the percentage coverage of the spinal cord lumbosacral enlargement by the scES paddle electrode. However, there were no significant correlations between the cross-section area of the spinal cord above the injury or the length of severe myelomalacia and these motor outcomes. This project is the first to investigate the relationship between volitional joint movements in the presence of scES and the positioning of the stimulation paddle electrode on the lumbosacral enlargement of the spinal cord using MRI and X-ray integration and the 3D volumetric reconstruction of the lumbosacral enlargement. The results herein suggest that positioning of scES paddle electrode to achieve the greatest lumbosacral enlargement coverage would facilitate voluntary recovery of movement in individuals with chronic clinically complete SCI. Moreover, the use of volumetric lumbosacral enlargement reconstruction pre-operatively provides a means to optimize placement of epidural stimulators for neuromodulation after spinal cord injury.

A second finding was that the amount of severe myelomalacia and cord atrophy above injury did not significantly correlate with the scES motor recovery. Previous studies have reported correlations between the amount of cord atrophy above the injury and extent of sensory and motor functions post injury in SCI cases when including both motor complete and incomplete SCI. Here, spinal cord atrophy did not significantly correlate with the functional recovery in the presence of scES when including only those with AIS A and B classifications. This may suggest that the return of lower extremity voluntary function can be achieved with scES in most cases even in the presence of severe post-traumatic radiographic abnormalities.

However, the variability across the research participants regarding the amount of neural tracts at the site of injury or expansion of the lesion to more distal normal-appearing spinal cord can potentially be contributing factors in regaining volitional recovery. Accurate quantification of the amount of residual fibers at the site of injury and measurement of abnormalities in more distal areas may unveil potential links to functional outcomes with scES. Yet, in the few studies that performed diffusion tensor imaging (DTI) and fiber tractography in clinically complete cases, the results of the tractography showed no fibers passing across the injury for AIS A while there were significant correlations between the DTI measures and AIS motor scores when including those with clinically incomplete SCI.

The observation that all research participants with diagnosed motor complete SCI in this study were able to move their joints voluntarily post implant may raise the question of whether these imaging methods have proper resolution to accurately detect the limited number of residual pathways that in the presence of epidural stimulation transmit the voluntary signal down to the spinal cord circuitry. In chronic and clinically complete SCI cases quantification of the amount of residual neural tracts at the site of injury using fiber tractography is often challenging due to the presence of surgical hardware that causes image artifacts at the injury site (in addition to respiratory and cerebrospinal fluid pulsation artifacts). Also, the small number of neural sparing that exist in AIS A and B cases can further limits detectability of these structures in the MRI recordings.

Functional MRI studies on clinically diagnosed motor complete SCI cases have revealed that in the presence of external motor and sensory stimuli, active dorsal and ventral networks within gray matter above and below the injury site and at lumbar spinal cord were observed even without sensation awareness. Cortical stimulation and recording of evoked muscle responses from paralyzed muscles also showed preserved motor pathways as well as existence of voluntary muscle responses in the presence of transcranial magnetic stimulation in most AIS A and B SCI cases studied and two with vestibular pathways observed across the injury level. The observation that all research participants in this study achieved some voluntary movement provides further evidence that direct neuromodulation of the lumbosacral spinal cord by scES enables the complex spinal networks to access the inputs from previously non-functional and non-detectable residual supraspinal fibers that can mediate intent. Thus, even after severe SCI, the descending and afferent signals can still reach the intact lumbosacral networks within the spinal cord via the remaining neural pathways; therefore, imaging and neurophysiological assessments need to be further developed and used synergistically for more predictive and mechanistic evaluation of neuroplasticity and recovery as new treatments emerge for SCI.

The achievement of voluntary movements prior to institution of post-operative locomotor training suggests that the findings of this study cannot be explained by Hebbian mechanisms involving a combination of scES and post-operative locomotor training. Previous studies including finite element modeling of spinal cord tissue structures and distribution patterns of electric field generated by scES suggested that dorsal roots have the lowest activation threshold and are the first structures to activate in the presence of scES. These studies also suggest that scES can indirectly activate the complex circuitry within the spinal cord through intersynaptic connections with posterior roots. Electrical stimulation of the lumbosacral spinal cord is therefore thought to enable volitional movements by modulating the propriospinal networks that regulate the interpretation of the volitional descending inputs as well as sensory information to generate desired motor patterns. Greater coverage of the lumbosacral enlargement by paddle electrode could mean that most of the propriospinal networks at lumbosacral level can directly be targeted by focused stimulation field at sub motor threshold intensities. Additional mechanisms of scES for enabling voluntary movements may include modulation of gene activity and synaptic plasticity at the lumbosacral level.

Clinical Significance. In the original epidural stimulation study of motor recovery, the research participants reported benefits to the autonomic function in bladder, sexual, and thermoregulatory activity. Subsequently epidural stimulation has expanded to many research and clinical sites and the benefits of scES have been reported on motor, bladder, bowel and cardiovascular function in a greater number but the extent of these benefits varies widely across individuals with SCI. Implementing the methodology used in this study may begin to explain some of this variability.

This disclosure establishes that the precise placement of the paddle electrode to maximize coverage of the lumbosacral enlargement is an important consideration for voluntary movement recovery in chronic, clinically diagnosed motor complete SCI. The novel approach disclosed herein uses clinically available techniques, fluoroscopy, X-ray, 3D MRI, and intra-operative neurophysiology to optimize scES paddle placement. The optimal design of the paddle electrode and contacts, synergistic imaging and neurophysiology assessments and its effectiveness to achieve the best recovery in SCI population has to be the subject of further investigation.

While the above disclosure identifies the advantages of positioning the implantable electrode paddle to maximize coverage of the of the lumbosacral enlargement, it remains challenging to do so during the initial positioning of the electrode paddle during implantation as the lumbosacral enlargement is surrounded by the vertebral column and not visible to the surgeon. While estimating the lumbosacral enlargement as the portion of the spinal cord extending the 2L distance from the conus tip is one method for locating the lumbosacral enlargement, it is not tied to specific spinal cord segments (also referred to as spinal cord segment levels or spinal cord levels) in the subject. Further work by the inventors has addressed this problem as well.

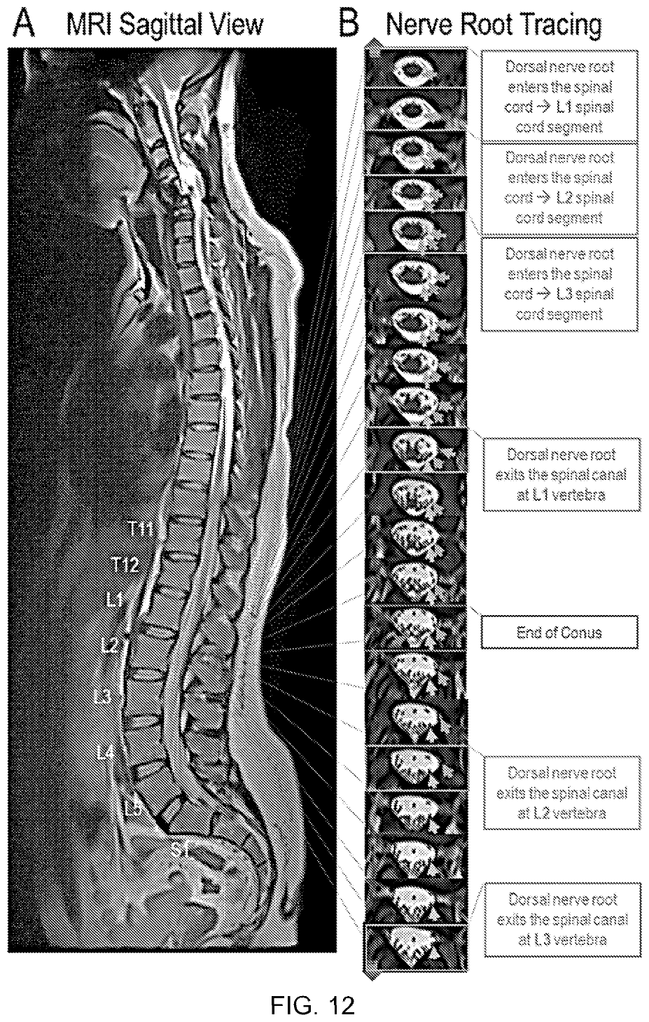

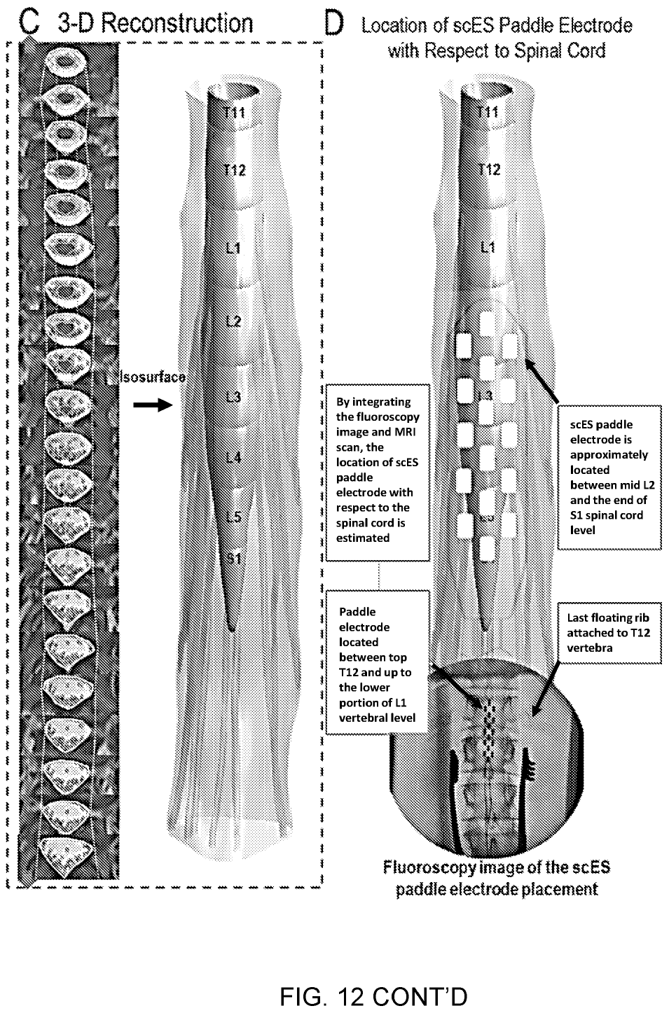

Spinal Cord Neuroanatomical Mapping at the Lumbosacral Enlargement (first step 18, second step 20, third step 22). Recording MRI axial scans with high spatial resolution (3 mm slice thickness and zero gap) were used to locate and trace the dorsal and ventral nerve roots that float in the cerebrospinal fluid. As the spinal cord typically ends at the L1 vertebral level, the nerve roots that enter the spinal cord at the lumbosacral enlargement start to elongate and exit the spinal canal further distally at the corresponding vertebral levels (L1, L2, . . . , S1). Therefore, by identifying the set of nerve roots (dorsal and ventral roots on the left and right sides) that exit the spinal canal at each vertebral level and back-tracing those nerve roots into the spinal cord body, the spinal cord lumbar segments assigned to L1, L2, . . . , S1 were anatomically estimated. The process of nerve root tracing and estimating spinal cord L1-S1 segments is referred to as spinal cord neuroanatomical mapping in this study and was performed manually by an expert analyst. Furthermore, the axial images of the lumbosacral spinal cord were traced and labeled based on the area of the cerebrospinal canal, spinal cord tissue, and nerve roots. A computational 3D model of the spinal cord of each individual was then reconstructed using custom-written software code in MATLAB. The estimated neuroanatomical levels of spinal cord were visualized on the 3D reconstructed model of the lumbosacral region (FIG. 12, Panels A-C). The total length of the spine and spinal cord, length and volume of the spinal cord and cerebrospinal canal at the lumbosacral enlargement and the conus area as well as the location of the conus tip with respect to the vertebral body were also measured and reported in order to measure the amount of variability across individuals. By comparing the spinal cord levels with the vertebral column in the computational model, one could position the paddle electrode on the vertebral column (which is visible during implantation surgery) at a position aligned with the lumbosacral enlargement (which is not visible during implantation surgery, and its location as compared to the vertebral column varies between individuals) to maximize coverage of the lumbosacral enlargement.

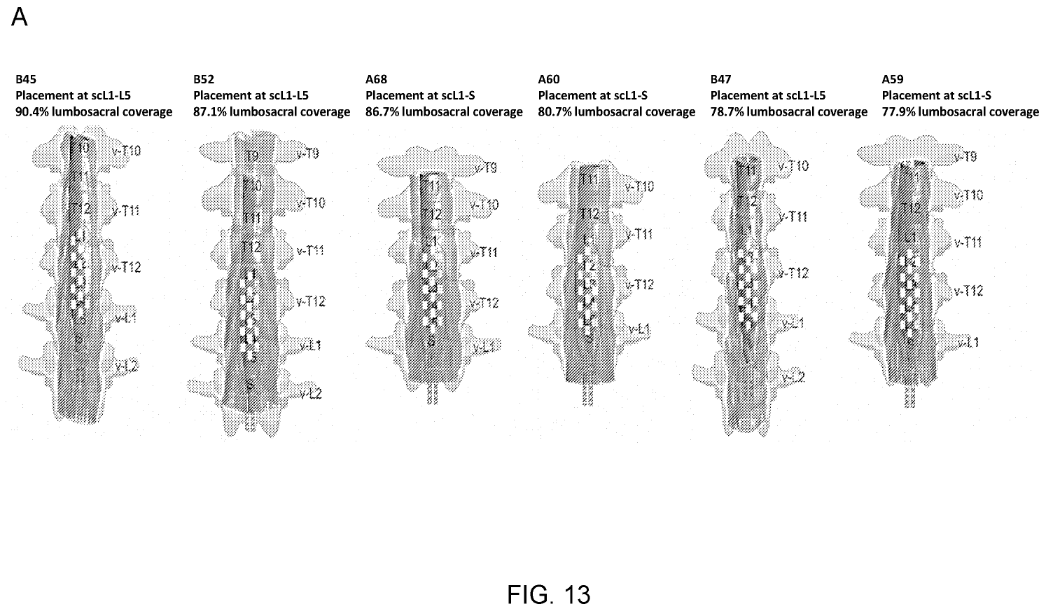

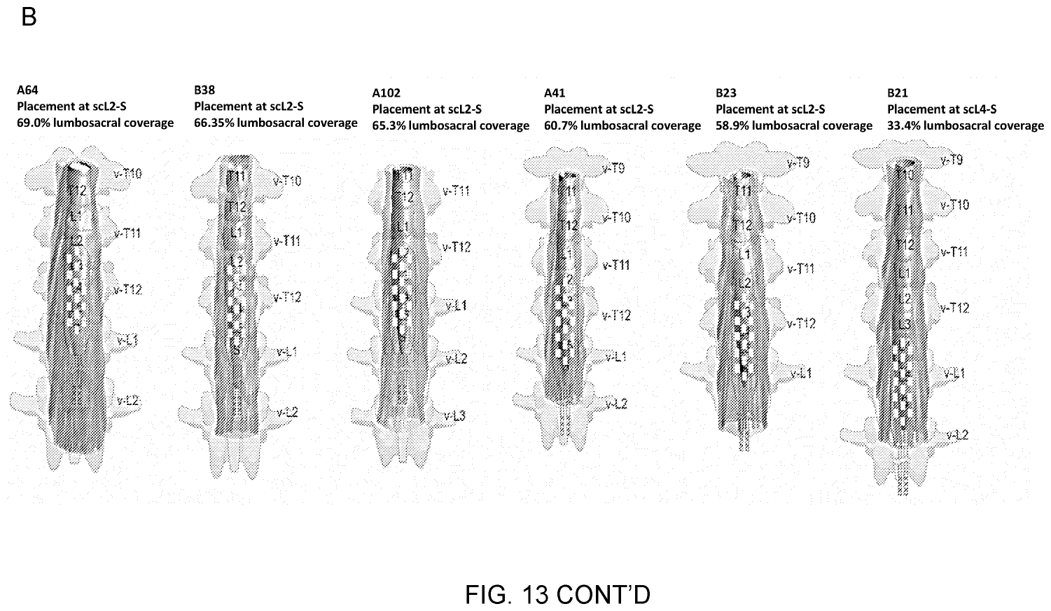

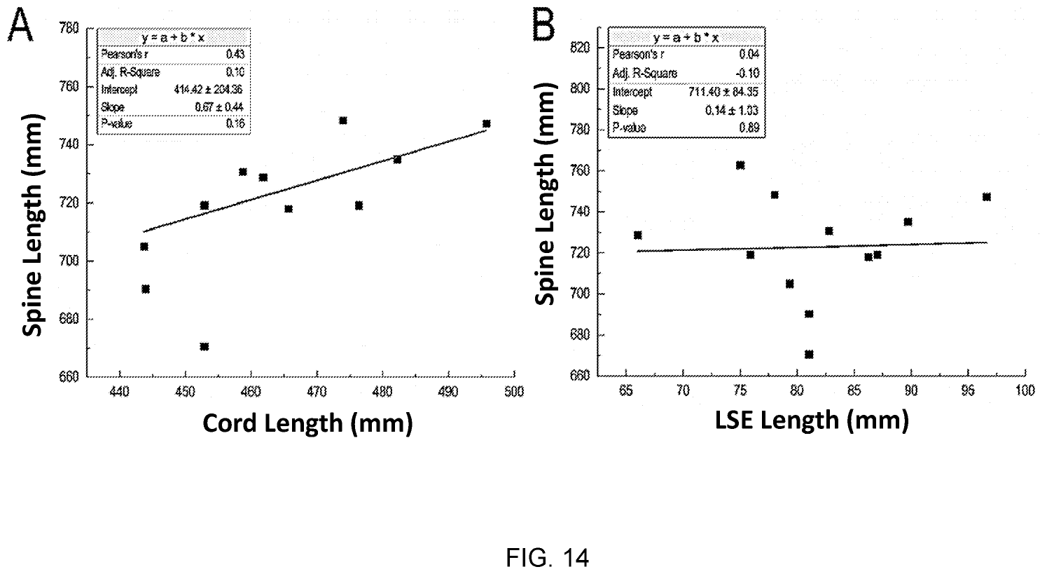

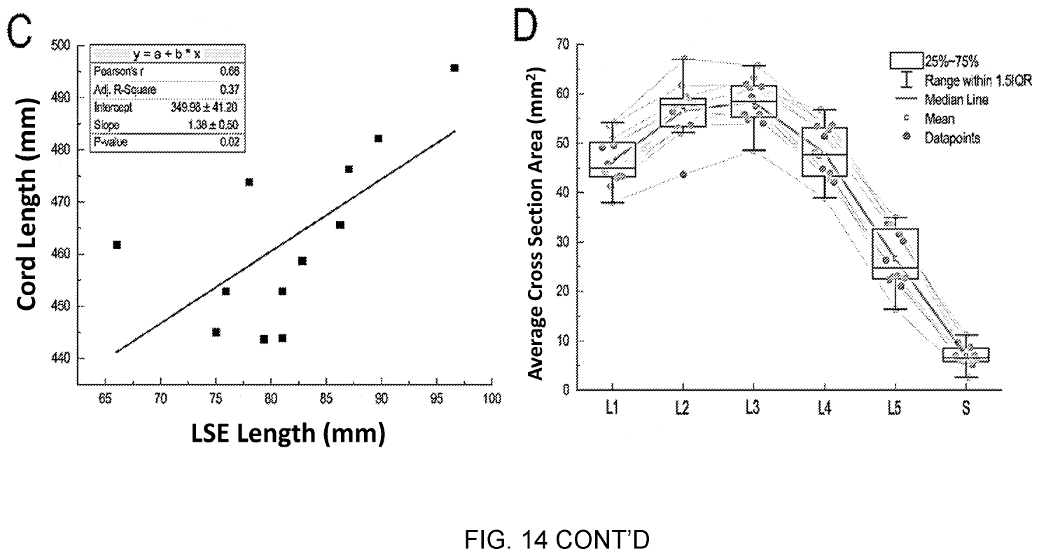

The size (length and volume) and location of the spinal cord segments with respect to the vertebral bodies at lumbosacral enlargement is highly variable across individuals (FIG. 13, panels A and B). The estimated location of spinal cord L1 segment (sc-L1) with respect to the vertebrae (v) ranges from v-T10/T11 gap to the end of v-T11 across individuals. Location of sc-L2 ranges from mid v-T11 to top of v-T12. Location of sc-L3 ranges from end of v-T11 to mid v-T12. Location of sc-L4 ranges from top of v-T12 to v-T12/L1 gap. Location of sc-L5 ranges from mid v-T12 to top of v-L1. Location of sc-S1 ranges from end of v-T12 to mid v-L1. Location of the tip of the conus ranges from top of v-L1 to mid L2. FIG. 14 provides correlations between total length of the spinal column, total length of the spinal cord and length of spinal cord lumbosacral enlargement as well as the boxplots of average cross-section areas of the spinal cord at L1, L2, L3, . . . , S1 levels. The correlation analysis show there are no statistically significant correlations between the length of the spinal column and total length of the spinal cord or the length of the lumbosacral enlargement (FIG. 14, Panels A and B). However, there was a significant moderate correlation between total length of the spinal cord and length of the lumbosacral enlargement (FIG. 14, Panel C).

The intra-operative fluoroscopy and post-operative X-ray images were used to identify the location of the paddle electrode with respect to the vertebrae (FIG. 12, Panel D; eighth step 34). By identifying the segmental locations of the spinal cord in relation to the vertebral bodies, the location of the paddle array with respect to the lumbosacral spinal cord was estimated. FIG. 13 illustrates the reconstructed spinal cord at lumbosacral enlargement with identified spinal cord levels via nerve root tracing and scES paddle placement overlaid on the 3D models for each participant in coronal view (n=12). The locations of spinal cord levels and conus tip with respect to the vertebrae are derived from the MRI scans and indicated with the illustrations of the vertebral bodies in these 3D models. These models visualize the amount of variability among individuals regarding the size and position of spinal cord levels with respect to the positioning of the scES implant as well as the location of encircled vertebrae. Volumetric coverage of the lumbosacral spinal cord ranges from 33.4% to 90.4% across participants.

The development of individual-specific spinal cord neuroanatomical mapping and 3D reconstruction modeling of the lumbosacral enlargement enables prediction of the optimum placement for each participant prior to the scES implantation surgery. This protocol as summarized in FIG. 1 allows for (i) identification of the anatomically optimal location for laminotomy for each participant, (ii) time reduction in intra-operative search for finding the best placement and (iii) identification of the conus tip to ensure full coverage of the spinal cord tissue thereby avoiding placement below the conus.

The analysis of the neuroanatomical characteristics of the spinal cord provided herein indicates that there is a considerable amount of variability across individuals regarding the volume and length of the spinal cord and enclosed vertebrae. The location of the end of the conus with respect to the vertebrae varies across individuals (ranges from top of v-L1 to mid v-L2). Similarly, variability exists among participants in the sc-L1, L2, . . . , S levels of lumbosacral region of the spinal cord with respect to surrounding vertebrae (FIG. 14). The findings also suggest that by only measuring the length of the spinal column in individuals, the length of lumbosacral enlargement cannot be estimated and having a longer (or shorter) spine does not necessarily indicate a longer (or shorter) spinal cord (FIG. 14, Panel A). The average values of the cross section areas of the spinal cord at sc-L1, L2, . . . , S levels across individuals (FIG. 14, Panel D, n=12) show sc-L3 has the largest spinal cord transverse diameter (directly related to the cross-section area) and indicates the area of maximal enlargement of the lumbosacral enlargement. These findings also show that the length of spinal cord from the top of lumbosacral enlargement (sc-L1) to the end of conus in all individuals ranges from 66.0 to 96.6 mm which is greater than the length of the current paddle electrode design (e.g., Medtronic Specify® 5-6-5 lead), 46.5 mm, thereby requiring compromise during surgical implantation to provide coverage of the spinal cord segments that are most essential for the individual and their most significant neurological deficits that need to be addressed with epidural stimulation.

Neurophysiological mapping of the human spinal cord to identify the topographical recruitment patterns of the lumbosacral enlargement that target activation of leg muscles has been performed previously using various experimental methodologies. Most of these studies have relied on the typical anatomical relations between spinal cord levels and vertebral levels that are reported in neuroanatomical literature rather than performing image-based nerve root tracing to identify the exact location of the lumbosacral levels of the spinal cord with respect to the bone. The findings herein suggest that the lack of the imaging component and information about the exact location of stimulation electrodes with respect to the spinal cord levels in these studies could potentially be a source of misinterpretation of the results of lumbosacral spinal cord functional mapping.

Variability in size (length, area, volume) lumbosacral spinal cord levels across individuals may result in differing mechanisms of action of scES in neuromodulation interventions among participants. The same electrode combinations may enable different neurophysiological outcomes across individuals due to targeting different spinal cord regions and activating various spinal cord networks. It should also be noted that other sources, such as extent, severity and mechanism of the injury, number of residual fibers as well as clinical and demographic factors may also influence the neuromodulatory effects of spinal cord stimulation and although maximizing coverage of the lumbosacral enlargement is important, it alone does not ensure full restoration of function after spinal cord injury.

Previous surgical implantation guidelines for epidural stimulation do not highlight the importance of a pre-implant medical image-based pre-planning approach. Here, a modified protocol for scES implantation surgery is presented that uses high-resolution imaging and pre-operative planning for optimum paddle placement to improve the implantation procedure and maximize coverage of the lumbosacral enlargement. The location of the lumbosacral enlargement may be determined by back-tracing nerve roots in medical images, which requires high-resolution imagining, or may be estimated based on the location on the spinal cord with maximal CSA as evidenced by medical images, which does not necessarily require high-resolution imaging. Coverage may be maximized in terms of volumetric coverage, i.e., maximizing the volume of the lumbosacral enlargement covered by the implanted electrode, or in terms of CSA coverage. Optimizing the paddle placement can also reduce the time in surgery for adjustments and intra-operative neurophysiological assessments which can potentially reduce complications such as, placements below conus, risk of infection and risks of repeated surgeries. An accurate knowledge of the location of the implant with respect to the spinal cord levels will also provide a vital tool for the researchers during post-implant mapping and stimulation-based training sessions that allows them to select the electrode combinations and intensities effectively based on an individual's spinal cord characteristics and allowing rigorous comparisons across individuals and studies which promotes understanding of the mechanism of action for modulating multiple systems.

Various aspects of different embodiments of the present disclosure are expressed in paragraphs X1 and X2 as follows:

X1. One embodiment of the present disclosure includes a method of optimizing electrode placement for spinal cord epidural stimulation, the method comprising receiving at least one medical image of a spinal cord and surrounding vertebral column; constructing, using a computer, a three-dimensional model of at least a portion of the spinal cord, the three-dimensional model based at least in part on the at least one medical image; determining the position of a lumbosacral enlargement of the spinal cord in the three-dimensional model based at least in part on the at least one medical image; and determining an optimal placement of an implantable electrode, wherein the optimal placement maximizes coverage of the lumbosacral enlargement by the implantable electrode.

X2. Another embodiment of the present disclosure includes a method of optimizing electrode placement for spinal cord epidural stimulation, the method comprising receiving at least one medical image of a spinal cord and surrounding vertebral column; identifying, based on the at least one medical image, at least one nerve root exiting the vertebral column and back-tracing the at least one nerve root to determine a position of at least one spinal cord segment; constructing a three-dimensional model of at least a portion of the spinal cord, the three-dimensional model based at least in part on the at least one medical image; determining the position of a lumbosacral enlargement of the spinal cord in the three-dimensional model based at least in part on the position of the at least one spinal cord segment; and determining an optimal placement of an implantable electrode, wherein the optimal placement maximizes coverage of the lumbosacral enlargement by the implantable electrode.

Yet other embodiments include the features described in any of the previous paragraphs X1 or X2, as combined with one of more of the following aspects:

Wherein determining the optimal placement includes identifying, using the three-dimensional model, a portion of the vertebral column surrounding the lumbosacral enlargement.

Wherein determining the position of the lumbosacral enlargement includes identifying, based on the at least one medical image, at least one nerve root exiting the vertebral column and back-tracing the at least one nerve root to determine a position of at least one spinal cord segment in the lumbosacral enlargement.

Wherein back-tracing the at least one nerve root to determine a position of at least one spinal cord segment includes determining the position of at least one L1-S1 spinal cord segments.

Wherein back-tracing the at least one nerve root to determine a position of at least one spinal cord segment includes determining the position of the L1-S1 spinal cord segments.

Wherein the at least one medical image is a plurality of axial images of the spinal cord captured at different vertebral levels.

Wherein the at least one medical image is a plurality of non-identical medical images.

Wherein the at least one medical image is a plurality of axial MRI images of the spinal cord captured at different vertebral levels.

Wherein the at least one medical image is a plurality of non-identical MRI images.

Wherein determining the position of the lumbosacral enlargement includes calculating a cross-section area of the lumbosacral enlargement in each of the plurality of axial images, identifying a location on the spinal cord having a maximal cross-section area based at least in part on the plurality of axial images, and determining the position of the lumbosacral enlargement based at least in part on the location on the spinal cord having the maximal cross-section area.

Including identifying, using the plurality of axial images, a conus tip of the spinal cord, and wherein determining the position of the lumbosacral enlargement is based at least in part on a distance between the conus tip and the location on the spinal cord having the maximal cross-section area.

Wherein the implantable electrode is a paddle electrode, and wherein optimal placement maximizes coverage of the lumbosacral enlargement by the paddle electrode.

Wherein coverage of the lumbosacral enlargement is determined by calculating a volume of the lumbosacral enlargement and determining a percentage of the volume of the lumbosacral enlargement overlaid by the paddle electrode.

Including identifying a position on the vertebral column corresponding to the position of the lumbosacral enlargement.

Wherein the three-dimensional model is a computational model.

The foregoing detailed description is given primarily for clearness of understanding and no unnecessary limitations are to be understood therefrom for modifications can be made by those skilled in the art upon reading this disclosure and may be made without departing from the spirit of the invention.

What is claimed is:

1) A method of optimizing electrode placement for spinal cord epidural stimulation, the method comprising:
receiving at least one medical image of a spinal cord and surrounding vertebral column;
constructing, using a computer, a three-dimensional model of at least a portion of the spinal cord, the three-dimensional model based at least in part on the at least one medical image;
determining the position of a lumbosacral enlargement of the spinal cord in the three-dimensional model based at least in part on the at least one medical image; and
determining an optimal placement of an implantable electrode, wherein the optimal placement maximizes coverage of the lumbosacral enlargement by the implantable electrode.

2) The method of claim 1, wherein determining the optimal placement includes identifying, using the three-dimensional model, a portion of the vertebral column surrounding the lumbosacral enlargement.

3) The method of claim 1, wherein determining the position of the lumbosacral enlargement includes identifying, based on the at least one medical image, at least one nerve root exiting the vertebral column and back-tracing the at least one nerve root to determine a position of at least one spinal cord segment in the lumbosacral enlargement.

4) The method of claim 3, wherein back-tracing the at least one nerve root to determine a position of at least one spinal cord segment includes determining the position of at least one L1-S1 spinal cord segment.

5) The method of claim 1, wherein the at least one medical image is a plurality of axial images of the spinal cord captured at different vertebral levels.

6) The method of claim 5, wherein determining the position of the lumbosacral enlargement includes calculating a cross-section area of the lumbosacral enlargement in each of the plurality of axial images, identifying a location on the spinal cord having a maximal cross-section area based at least in part on the plurality of axial images, and determining the position of the lumbosacral enlargement based at least in part on the location on the spinal cord having the maximal cross-section area.

7) The method of claim 6, further comprising identifying, using the plurality of axial images, a conus tip of the spinal cord, and wherein determining the position of the lumbosacral enlargement is based at least in part on a distance between the conus tip and the location on the spinal cord having the maximal cross-section area.

8) The method of claim 1, wherein the implantable electrode is a paddle electrode, and wherein optimal placement maximizes coverage of the lumbosacral enlargement by the paddle electrode.

9) The method of claim 8, wherein coverage of the lumbosacral enlargement is determined by calculating a volume of the lumbosacral enlargement and determining a percentage of the volume of the lumbosacral enlargement overlaid by the paddle electrode.

10) The method of claim 1, further comprising identifying a position on the vertebral column corresponding to the position of the lumbosacral enlargement.

11) A method of optimizing electrode placement for spinal cord epidural stimulation, the method comprising:
receiving at least one medical image of a spinal cord and surrounding vertebral column;
identifying, based on the at least one medical image, at least one nerve root exiting the vertebral column and back-tracing the at least one nerve root to determine a position of at least one spinal cord segment;
constructing a three-dimensional model of at least a portion of the spinal cord, the three-dimensional model based at least in part on the at least one medical image;
determining the position of a lumbosacral enlargement of the spinal cord in the three-dimensional model based at least in part on the position of the at least one spinal cord segment; and
determining an optimal placement of an implantable electrode, wherein the optimal placement maximizes coverage of the lumbosacral enlargement by the implantable electrode.

12) The method of claim 11, wherein the at least one medical image is a plurality of non-identical medical images.

13) The method of claim 11, wherein the at least one medical image is a plurality of axial MRI images of the spinal cord captured at different vertebral levels.

14) The method of claim 11, wherein back-tracing the at least one nerve root to determine a position of at least one spinal cord segment includes determining the position of at least one L1-S1 spinal cord segment.

15) The method of claim 14, wherein back-tracing the at least one nerve root to determine a position of at least one spinal cord segment includes determining the position of the L1-S1 spinal cord segments.

16) The method of claim 11, wherein the implantable electrode is a paddle electrode, and wherein optimal placement maximizes coverage of the lumbosacral enlargement by the paddle electrode.

17) The method of claim 16, wherein coverage of the lumbosacral enlargement is determined by calculating a volume of the lumbosacral enlargement and determining a percentage of the volume of the lumbosacral enlargement overlaid by the paddle electrode.

18) The method of claim 11, further comprising identifying a position on the vertebral column corresponding to the position of the lumbosacral enlargement.

19) The method of claim 11, wherein the three-dimensional model is a computational model.
